(19) United States
(12) Patent Application Publication
(10) Pub. No.: US 2021/0127678 A1
(43) Pub. Date: May 6, 2021

(54) ESTERS OF HETEROCYCLIC COMPOUNDS HAVING A NEMATOCIDAL ACTIVITY, THEIR AGRONOMIC COMPOSITIONS AND THEIR USE

(71) Applicant: ISAGRO, S.P.A., Milan (IT)

(72) Inventors: Giuseppe D'ORAZIO, Bollate (MI) (IT); Serena CARIELLO, Novara (NO) (IT); Marilena GUSMEROLI, Monza (MB) (IT); Paolo BELLANDI, Carcare (SV) (IT); Chiara SARGIOTTO, Galliate (NO) (IT); Daniele BIANCHI, Bellinzago Novarese (NO) (IT); Riccardo LIGUORI, Monza (MB) (IT)

(73) Assignee: Isagro S.p.A., Milan (IT)

(21) Appl. No.: 17/041,919

(22) PCT Filed: Mar. 29, 2019
(86) PCT No.: PCT/IB2019/052617

§ 371 Date: Sep. 25, 2020

(30) Foreign Application Priority Data
Mar. 30, 2018 (IT) 102018000004120

(51) Int. Cl. A01N 43/78; A01N 43/82; C07D 277/56; C07D 285/12

(52) U.S. Cl. CPC A01N 43/78; C07D 285/12; C07D 277/56; A01N 43/82

(57) ABSTRACT

Fluoroalkenyl heterocyclic compounds having Formula (I) are described together with agronomic compositions containing said compounds having formula (I) and their use for the control of nematodes in agricultural crops.

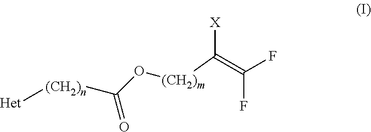

The present invention relates to new fluoroalkenyl heterocyclic compounds having Formula (I)

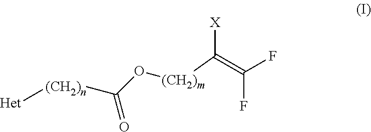

The present invention also relates to agronomic compositions which contain said compounds having formula (I) and their use for the control of nematodes in agricultural crops.

### STATE OF THE ART

Esters of fluoroalkenyl heterocyclic compounds have been described in literature for use as pesticides and, in particular, as nematocides.

These compounds consist of three characteristic residues: an aromatic heterocycle, a —(CH2)ₙ—COO— residue wherein n is an integer from 0 to 2 and a fluoroalkenyl chain having general formula —(CH₂)ₘ—CX═CF₂ wherein m can vary from 1 to 6 and X is a hydrogen atom or a halogen.

Patent application JP2000/038379 describes pyridine ester compounds substituted in position 2, 3 or 4 with a fluoroalkenyl chain and suitable substituents on the ring.

Patent application JP2000/086636 describes pyrazole derivatives carrying the above-mentioned fluoroalkenyl chain.

Other heterocyclic groups such as thiophen-2-yl, furan-2-yl, pyrazin-2-yl, quinol-4-yl or pyrrol-2-yl are described in patent application JP2000/186073, substituted with the same fluoroalkenyl chain.

All of these compounds however have proved to be unsatisfactory in terms of nematocidal activity, as they cannot effectively limit the attack of the parasite and reduce the formation of galls on the root system of the plant.

Furthermore, in many cases, these products are phytotoxic with respect to important agricultural crops at doses that allow a good nematocidal activity to be obtained, showing a significant necrosis of the leaves and stem.

### DESCRIPTION

The Applicant has now surprisingly found new esters of fluoroalkenyl heterocyclic compounds which overcome the drawbacks outlined above, characterized by a high nematocidal activity even at low doses and which, at the same time, are well tolerated by agricultural crops.

An object of the present invention therefore relates to esters of heterocyclic compounds having general formula (I):

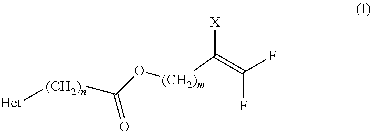

wherein:
Het represents an aromatic heterocycle having from 5 to 10 terms, possibly benzo-fused, or heterobicyclic containing heteroatoms selected from nitrogen, sulfur, and oxygen; said heterocycle being optionally substituted by one or more Y groups selected from halogen, C₁-C₆-alkyl, C₁-C₆-haloalkyl, C₃-C₆-cycloalkyl, C₄-C₇-cycloalkylalkyl, C₁-C₆-alkoxyl, C₁-C₆-halo alkoxyl, C₁-C₆-thioalkoxyl, C₁-C₆-thiohaloalkoxyls, C₁-C₆-alkylsulfinyl, C₁-C₆-alkylsulfonyl, C₁-C₆-alkoxycarbonyl, C₃-C₆-cycloalkoxycarbonyl, amino, N—C₁-C₆-alkylamino, N,N—C₂-C₁₂-dialkylamino, N—C₁-C₆-alkoxycarbonylamino, N—C₃-C₆-cycloalkylamino, N,N—C₆-C₁₂-dicycloalkylamino, N—C₃-C₆-cycloalkoxycarbonylamino, C₁-C₆-alkylaminocarbonyl, C₃-C₆-cycloalkylaminocarbonyl, a NR₁R₂CONR₁-formyl group, C₁-C₆-alkylcarbonyl, carboxyl, cyano, an aryl, optionally substituted, a benzyl, an aromatic heterocycle, optionally substituted, penta- or hexa-atomic optionally benzo-fused or heterobicyclic, containing at least one heteroatom selected from oxygen, sulfur, nitrogen, possibly oxidized to N-oxide;
n represents an integer ranging from 0 to 4;
m represents an integer ranging from 1 to 6;
X represents an H or fluorine atom;
R₁, R₂ the same or different, represent a hydrogen atom, a C₁-C₄ alkyl group or a C₃-C₆-cycloalkyl group,

with the proviso that:

if n has the value of 1, X represents an F atom for any meaning of Het and m;

if Het represents a thiophen-2-yl group or a furan-2-yl group or a pyrazin-2-yl group or a quinol-4-yl or pyrrolyl-2-yl group, substituted or not substituted by one or more Y groups or Het represents a pyrazol-5-yl group or a pyridyl-2-yl or pyridyl-3-yl or pyridyl-4-yl group not substituted or substituted by one or more Y groups selected from halogen, C₁-C₆-alkyl, C₁-C₆-alkoxyl, C₁-C₆-thioalkoxyl, C₁-C₆-thiohaloalkoxyls, aryl, or Het represents a 1,2,3-thiadiazol-4-yl group or a 1,2,3-thiadiazol-5-yl group substituted or not substituted by one or more Y groups, then, contemporaneously, n must be different from 0 and X different from H;

if Het represents a pyrazol-4-yl group substituted or not substituted by one or more Y groups, then contemporaneously n must be different from 1, m different from 1 and X different from F.

Examples of Het are: thiazolyl, 1,3,4-thiazolyl, pyrimidyl, imidazolyl, indolyl, benzothiazolyl, benzofuranyl, [1,2,4]-triazole-[3,2-b]-thiazolyl.

Examples of halogen are: fluorine, chlorine, bromine, iodine.

Examples of linear or branched C₁-C₆ alkyl are methyl, ethyl, n-propyl, isopropyl, n-butyl, isobutyl, sec-butyl, tert-butyl, n-pentyl, 3-methylbutyl, n-hexyl, 3,3-dimethylbutyl.

Examples of C₁-C₆ haloalkyl are fluoromethyl, difluoromethyl, trifluoromethyl, chloromethyl, dichloromethyl, 2,2,2-trifluoroethyl, 1,1,2,2-tetrafluoroethyl, pentafluoroethyl, heptafluoropropyl, 4,4,4-trichloro-butyl, 4,4-difluoropentyl, 5,5-difluorohexyl.

Examples of linear or branched C₁-C₆ alkoxyl are methoxy, ethoxy, n-propoxy, isopropoxy, n-butoxy, isobutoxy, sec-butoxy, tert-butoxy, n-pentoxy, 3-methylbutoxy, hexyloxy, 3,3-dimethylbutoxy.

Examples of C₁-C₆ haloalkoxyl are fluoromethoxy, difluoromethoxy, trifluoromethoxy, chloromethoxy, dichlorome-thoxy, 2,2,2-trifluoroethoxy, 1,1,2,2-tetra-fluoroethoxy, 1,1,2,3,3,3-hexafluoropropoxy, 4,4,4-trichlorobutoxy, 4,4-difluoropentoxy, 5,5-difluorohexyloxy.

Examples of C₃-C₆ cycloalkyl are cyclopropyl, cyclobutyl, cyclopentyl, cyclohexyl. Examples of C₃-C₈ halocycloalkyl are 2,2-dichlorocyclopropyl, 2,2-difluorocyclopropyl, 2,2,3,3-tetrafluo-rocyclobutyl, 3,3-difluorocyclopentyl, 2-fluorocyclohexyl.

Examples of C₄-C₇ cycloalkylalkyl are methylcyclopropyl, methylcyclopentyl, ethylcyclopropyl, methylcyclohexyl.

Aryl optionally substituted refers to an aryl group that can have one or more substituents, the same as or different from each other, preferably selected from the following groups: halogen atoms, C₁-C₆ alkyls, C₁-C₆ haloalkyls, C₁-C₆ alkoxyls, C₁-C₆ haloalkoxyls, C₃-C₆ cycloalkoxyls, C₄-C₇ cycloalkylalkoxyls, phenoxyls, C₁-C₆ alkylthio, C₁-C₆ haloalkylthio, C₁-C₆ alkylsulfinyls, C₁-C₆ alkylsulfonyls, C₁-C₆ alkylamino, C₂-C₁₂ dialkylamino, cyano, C₂-C₇ alkoxycarbonyls, benzyloxy-carbonyls, phenoxycarbonyls.

Aromatic heterocycle optionally substituted refers to a penta- or hexa-atomic heterocyclic ring, optionally benzo-fused or heterobicyclic, containing at least one heteroatom selected from nitrogen, oxygen, sulfur and that can have one or more substituents, the same as or different from each other, preferably selected from the following groups: halogen atoms, C₁-C₆ alkyls, C₁-C₆ haloalkyls, C₁-C₆ alkoxyls, C₁-C₆ haloalkoxyls, C₃-C₆ cycloalkoxyls, C₄-C₇ cycloalkylalkoxyls, phenoxyls, C₁-C₆ thioalkoxyls, C₁-C₆ thiohaloalkoxyls, C₁-C₆ alkylsulfinyls, C₁-C₆ alkylsulfonyls, amino possibly substituted by one or two C₁-C₆ alkyl groups, cyano, nitro, carboxyl, C₂-C₅ alkoxycarbonyls, benzyloxycarbonyls, phenoxycarbonyls.

Examples of heteroaryl groups are: pyridine, pyridine N-oxide, pyrimidine, pyridazine, pyrazine, furan, thiophene, pyrrole, oxazole, thiazole, isoxazole, isothiazole, oxadiazole, thiadiazole, pyrazole, imidazole, triazole, indole, benzofuran, benzothiophene, benzoxazole, benzothiazole, benzoxadiazole, benzothiadiazole, benzopyrazole, benzimidazole, benzotriazole, triazolopyridine, triazolopyrimidine, thiazole.

The following also fall within the scope of the present invention:

a) all possible geometric isomers of the compounds having general formula (I);

b) the salts of the compounds having formula (I) obtained by the addition of inorganic or organic acids;

c) any hydrated forms of the compounds having formula (I).

Specific examples of compounds having general formula (I) preferred for their nematocidal activity are compounds wherein Het, n, m, and X have the meanings indicated in Table 1:

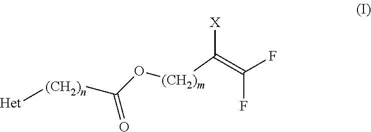

TABLE 1

| Comp. Nr. | Het | n | m | X |
|---|---|---|---|---|
| 1 | 5-Cl-thiazol-2-yl | 0 | 2 | F |
| 2 | 2-CH₃-4-CF₂H-thiazol-5-yl | 0 | 2 | F |
| 3 | 2-CH₃-4-CF₃-thiazol-5-yl | 0 | 2 | F |
| 4 | 5-CH₃-1,3,4-thiadiazol-2-yl | 0 | 2 | F |
| 5 | 5-CH₃-1,3,4-oxadiazol-2-yl | 0 | 2 | F |
| 6 | 5-iPr-1,3-oxazol-4-yl | 0 | 2 | F |
| 7 | 2-CH₃-5-CF₃-oxazol-4-yl | 0 | 2 | F |
| 8 | 2-CF₃-thiazol-4-yl | 0 | 2 | F |
| 9 | 2-iPr-thiazol-4-yl | 0 | 2 | F |
| 10 | 2-iPr-4-CH₃-1,3-thiazol-5-yl | 0 | 2 | F |
| 11 | Oxazol-5-yl | 0 | 2 | F |
| 12 | 2-CF₃-pyridyl-3-yl | 0 | 2 | F |
| 13 | 1-CH₃-3-CF₂H-pyrazol-4-yl | 0 | 2 | F |
| 14 | 2-Cl-furan-3-yl | 0 | 2 | F |
| 15 | 5-iPr-1,3,4-thiadiazol-2-yl | 0 | 2 | F |
| 16 | 1-CH₃-2-CH₃-5-CH₃-pyrazol-4-yl | 0 | 2 | F |
| 17 | 6-CF₃-8-Cl-imidazo-[1,2-a]-pyridyl-2-yl | 0 | 2 | F |
| 18 | Benzothiazol-2-yl | 0 | 2 | F |
| 19 | 5-CH₃-benzothiazol-2-yl | 0 | 2 | F |
| 20 | Benzothien-2-yl | 0 | 2 | F |
| 21 | Benzofuran-2-yl | 0 | 2 | F |
| 22 | 5-CH₃-[1,2,4]-triazole-[3,2-b]-thiazol-6-yl | 1 | 2 | F |
| 23 | 2-CH₃-thiazol-5-yl | 1 | 2 | F |
| 24 | 2-CH₃-4-CH₃-thiazol-5-yl | 1 | 2 | F |
| 25 | 2-CH₃-4-CF₂H-thiazol-5-yl | 0 | 4 | F |
| 26 | 2-CH₃-4-CF₃-thiazol-5-yl | 0 | 4 | F |
| 27 | 5-CH₃-1,3,4-thiadiazol-2-yl | 0 | 4 | F |
| 28 | 5-CH₃-1,3,4-oxadiazol-2-yl | 0 | 4 | F |
| 29 | 5-iPr-1,3-oxazol-4-yl | 0 | 4 | F |
| 30 | 2-CH₃-5-CF₃-oxazol-4-yl | 0 | 4 | F |
| 31 | 2-CF₃-thiazol-4-yl | 0 | 4 | F |
| 32 | 2-iPr-thiazol-4-yl | 0 | 4 | F |
| 33 | 2-iPr-4-CH₃-1,3-thiazol-5-yl | 0 | 4 | F |
| 34 | Oxazol-5-yl | 0 | 4 | F |
| 35 | 2-CF₃-pyridyl-3-yl | 0 | 4 | F |
| 36 | 1-CH₃-3-CF₂H-pyrazol-4-yl | 0 | 4 | F |
| 37 | 2-Cl-furan-3-yl | 0 | 4 | F |
| 38 | 5-iPr-1,3,4-thiadiazol-2-yl | 0 | 4 | F |
| 39 | 1-CH₃-2-CH₃-5-CH₃-pyrazol-4-yl | 0 | 4 | F |
| 40 | 6-CF₃-8-Cl-imidazo-[1,2-a]-pyridyl-2-yl | 0 | 4 | F |
| 41 | Benzothiazol-2-yl | 0 | 4 | F |
| 42 | 5-CH₃-benzothiazol-2-yl | 0 | 4 | F |
| 43 | Benzothien-2-yl | 0 | 4 | F |
| 44 | Benzofuran-2-yl | 0 | 4 | F |
| 45 | 5-CH₃-[1,2,4]-triazole-[3,2-b]-thiazol-6-yl | 1 | 4 | F |
| 46 | 2-CH₃-thiazol-5-yl | 1 | 4 | F |
| 47 | 2-CH₃-4-CH₃-thiazol-5-yl | 1 | 4 | F |
| 48 | 2-CH₃-4-CF₂H-thiazol-5-yl | 0 | 4 | F |
| 49 | 2-CH₃-4-CF₃-thiazol-5-yl | 0 | 4 | F |
| 50 | 5-CH₃-1,3,4-thiadiazol-2-yl | 0 | 4 | F |
| 51 | 5-CH₃-1,3,4-oxadiazol-2-yl | 0 | 4 | F |
| 52 | 2-CF₃-pyridyl-3-yl | 0 | 4 | F |
| 53 | 5-iPr-1,3-oxazol-4-yl | 0 | 4 | F |
| 54 | 2-CH₃-5-CF₃-oxazol-4-yl | 0 | 4 | F |
| 55 | 2-CF₃-thiazol-4-yl | 0 | 4 | F |
| 56 | 2-iPr-thiazol-4-yl | 0 | 4 | F |
| 57 | 2-iPr-4-CH₃-1,3-thiazol-5-yl | 0 | 4 | F |
| 58 | Oxazol-5-yl | 0 | 4 | F |
| 59 | 1-CH₃-3-CF₃-pyrazol-4-yl | 0 | 4 | F |
| 60 | 2-Cl-furan-3-yl | 0 | 4 | F |
| 61 | 5-iPr-1,3,4-thiadiazol-2-yl | 0 | 4 | F |
| 62 | 1-CH₃-2-CH₃-5-CH₃-pyrazol-4-yl | 0 | 4 | F |
| 63 | 6-CF₃-8-Cl-imidazo-[1,2-a]-pyridyl-2-yl | 0 | 4 | F |
| 64 | Benzothiazol-2-yl | 0 | 4 | F |
| 65 | 5-CH₃-benzothiazol-2-yl | 0 | 4 | F |
| 66 | Benzothien-2-yl | 0 | 4 | F |
| 67 | Benzofuran-2-yl | 0 | 4 | F |
| 68 | 5-CH₃-[1,2,4]-triazole-[3,2-b]-thiazol-6-yl | 1 | 4 | F |
| 69 | 2-CH₃-thiazol-5-yl | 1 | 4 | F |
| 70 | 2-CH₃-4-CH₃-thiazol-5-yl | 1 | 4 | F |
| 71 | 2-CH₃-4-CF₂H-thiazol-5-yl | 0 | 2 | H |
| 72 | 2-CH₃-4-CF₃-thiazol-5-yl | 0 | 2 | H |
| 73 | 5-CH₃-1,3,4-thiadiazol-2-yl | 0 | 2 | H |
| 74 | 5-CH₃-1,3,4-oxadiazol-2-yl | 0 | 2 | H |
| 75 | 5-iPr-1,3-oxazol-4-yl | 0 | 2 | H |
| 76 | 2-CH₃-5-CF₃-oxazol-4-yl | 0 | 2 | H |
| 77 | 2-CF₃-thiazol-4-yl | 0 | 2 | H |
| 78 | 2-iPr-thiazol-4-yl | 0 | 2 | H |
| 79 | 2-iPr-4-CH₃-1,3-thiazol-5-yl | 0 | 2 | H |
| 80 | Oxazol-5-yl | 0 | 2 | H |
| 81 | 2-CF₃-pyridyl-3-yl | 0 | 2 | H |
| 82 | 1-CH₃-3-CF₂H-pyrazol-4-yl | 0 | 2 | H |
| 83 | 2-Cl-furan-3-yl | 0 | 2 | H |
| 84 | 5-iPr-1,3,4-thiadiazol-2-yl | 0 | 2 | H |
| 85 | 1-CH₃-2-CH₃-5-CH₃-pyrazol-4-yl | 0 | 2 | H |
| 86 | 6-CF₃-8-Cl-imidazo-[1,2-a]-pyridyl-2-yl | 0 | 2 | H |
| 87 | Benzothiazol-2-yl | 0 | 2 | H |
| 88 | 5-CH₃-benzothiazol-2-yl | 0 | 2 | H |
| 89 | Benzothien-2-yl | 0 | 2 | H |
| 90 | Benzofuran-2-yl | 0 | 2 | H |
| 91 | 1-CH₃-3-CF₂H-pyrazol-4-yl | 0 | 4 | H |
| 92 | 1-CH₃-3-CF₂H-pyrazol-4-yl | 0 | 1 | F |
| 93 | 1-CH₃-3-CF₂H-pyrazol-4-yl | 0 | 1 | H |
| 94 | 2-CF₃-pyridyl-3-yl | 0 | 4 | H |
| 95 | 2-CF₃-pyridyl-3-yl | 0 | 1 | F |
| 96 | 2-CF₃-pyridyl-3-yl | 0 | 1 | H |
| 97 | 2-CH₃-4-CF₂H-thiazol-5-yl | 0 | 4 | H |
| 98 | 2-CH₃-4-CF₃-thiazol-5-yl | 0 | 4 | H |
| 99 | 5-CH₃-1,3,4-thiadiazol-2-yl | 0 | 4 | H |
| 100 | 5-CH₃-1,3,4-oxadiazol-2-yl | 0 | 4 | H |
| 101 | 5-iPr-1,3-oxazol-4-yl | 0 | 2 | H |
| 102 | 2-CH₃-5-CF₃-oxazol-4-yl | 0 | 2 | H |
| 103 | 2-CF₃-thiazol-4-yl | 0 | 2 | H |
| 104 | 2-iPr-thiazol-4-yl | 0 | 2 | H |
| 105 | 2-iPr-4-CH₃-1,3-thiazol-5-yl | 0 | 2 | H |
| 106 | Oxazol-5-yl | 0 | 2 | H |
| 107 | 2-CF₃-pyridyl-3-yl | 0 | 2 | H |
| 108 | 1-CH₃-3-CF₃-pyrazol-4-yl | 0 | 2 | H |
| 109 | 2-Cl-furan-3-yl | 0 | 2 | H |
| 110 | 5-iPr-1,3,4-thiadiazol-2-yl | 0 | 2 | H |
| 111 | 1-CH₃-2-CH₃-5-CH₃-pyrazol-4-yl | 0 | 2 | H |
| 112 | 6-CF₃-8-Cl-imidazo-[1,2-a]-pyridyl-2-yl | 0 | 2 | H |
| 113 | Benzothiazol-2-yl | 0 | 2 | H |
| 114 | 5-CH₃-benzothiazol-2-yl | 0 | 2 | H |
| 115 | Benzothien-2-yl | 0 | 2 | H |
| 116 | Benzofuran-2-yl | 0 | 2 | H |
| 117 | 5-Cl-thiazol-2-yl | 0 | 2 | H |
| 118 | 1-CH₃-3-CF₃-pyrazol-4-yl | 0 | 1 | F |
| 119 | 1-CH₃-3-CF₃-pyrazol-4-yl | 0 | 1 | H |
| 120 | 1-CH₃-3-CF₃-pyrazol-4-yl | 0 | 4 | H |
| 121 | 1-CH₃-3-CF₃-pyrazol-4-yl | 0 | 2 | F |
| 122 | 2-Ph-4-CH₃-1,3-thiazol-5-yl | 0 | 2 | F |
| 123 | 6-CF₃-pyridin-3-yl | 0 | 2 | F |
| 124 | 2-CF₃-6-Cl-pyridin-3-yl | 0 | 2 | F |
| 125 | 4-CF₃-pyridin-3-yl | 0 | 2 | F |
| 126 | Pyrimidin-2-yl | 0 | 2 | F |
| 127 | Pyrimidin-5-yl | 0 | 2 | F |
| 128 | Pyrimidin-4-yl | 0 | 2 | F |
| 129 | 5-Me-1,2-oxazol-3-yl | 0 | 2 | F |
| 130 | 4,5-diCl-1,2-thiazol-3-yl | 0 | 2 | F |
| 131 | Pyridin-3-yl | 2 | 2 | F |
| 132 | Pyridin-3-yl | 1 | 2 | F |
| 133 | 6-CF₃-pyridin-3-yl | 1 | 2 | F |
| 134 | 6-CF₃-pyridin-3-yl | 2 | 2 | F |
| 135 | 4-CF₃-pyridin-3-yl | 2 | 2 | F |
| 136 | Pyrimidin-2-yl | 1 | 2 | F |
| 137 | Pyrimidin-5-yl | 1 | 2 | F |
| 138 | Pyrimidin-4-yl | 1 | 2 | F |
| 139 | Pyrimidin-2-yl | 2 | 2 | F |
| 140 | Pyrimidin-5-yl | 2 | 2 | F |
| 141 | Pyrimidin-4-yl | 2 | 2 | F |

Particularly preferred are compounds having general formula (I) wherein Het, n, m and X have the meanings indicated in Table 2.

TABLE 2

| Comp. Nr. | Het | n | m | X |
|---|---|---|---|---|
| 2 | 2-CH₃-4-CF₂H-thiazol-5-yl | 0 | 2 | F |
| 3 | 2-CH₃-4-CF₃-thiazol-5-yl | 0 | 2 | F |
| 4 | 5-CH₃-1,3,4-thiadiazol-2-yl | 0 | 2 | F |
| 5 | 5-CH₃-1,3,4-oxadiazol-2-yl | 0 | 2 | F |
| 6 | 5-iPr-1,3-oxazol-4-yl | 0 | 2 | F |
| 7 | 2-CH₃-5-CF₃-oxazol-4-yl | 0 | 2 | F |
| 8 | 2-CF₃-thiazol-4-yl | 0 | 2 | F |
| 9 | 2-iPr-thiazol-4-yl | 0 | 2 | F |
| 10 | 2-iPr-4-CH₃-1,3-thiazol-5-yl | 0 | 2 | F |
| 11 | Oxazol-5-yl | 0 | 2 | F |
| 12 | 2-CF₃-pyridyl-3-yl | 0 | 2 | F |
| 13 | 1-CH₃-3-CF₂H-pyrazol-4-yl | 0 | 2 | F |
| 14 | 2-Cl-furan-3-yl | 0 | 2 | F |
| 15 | 5-iPr-1,3,4-thiadiazol-2-yl | 0 | 2 | F |
| 16 | 1-CH₃-2-CH₃-5-CH₃-pyrazol-4-yl | 0 | 2 | F |
| 17 | 6-CF₃-8-Cl-imidazo-[1,2-a]-pyridyl-2-yl | 0 | 2 | F |
| 18 | Benzothiazol-2-yl | 0 | 2 | F |
| 19 | 5-CH₃-benzothiazol-2-yl | 0 | 2 | F |
| 20 | Benzothien-2-yl | 0 | 2 | F |
| 21 | Benzofuran-2-yl | 0 | 2 | F |
| 92 | 1-CH₃-3-CF₂H-pyrazol-4-yl | 0 | 1 | F |
| 95 | 2-CF₃-pyridyl-3-yl | 0 | 1 | F |
| 118 | 1-CH₃-3-CF₃-pyrazol-4-yl | 0 | 1 | F |
| 121 | 1-CH₃-3-CF₃-pyrazol-4-yl | 0 | 2 | F |
| 127 | Pyrimidin-5-yl | 0 | 2 | F |
| 129 | 5-Me-1,2-oxazol-3-yl | 0 | 2 | F |
| 131 | Pyridin-3-yl | 2 | 2 | F |

The compounds having general formula (I) can be prepared starting from the corresponding heterocyclic acid having general formula (II) by esterification reaction with a suitable alcohol having general formula (III) as indicated in reaction scheme 1, according to methods well known in organic chemistry.

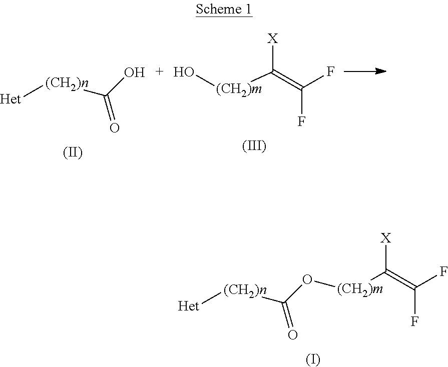

The reaction conditions provide for the use of a condenser such as, for example, N,N-dicyclohexylcarbodiimide, in the presence or absence of an amine such as N,N-dimethylaminopyridine, in an appropriate solvent such as dichloromethane, chloroform, tetrahydrofuran or dioxane, at a temperature ranging from 0° C. to the boiling point of the solvent, for a time ranging from 1 to 72 hours.

The compounds having formula (I) can also be obtained by reaction of the acid having formula (II) with the alcohol having formula (III) in the presence of an acid catalysis, using for example hydrochloric acid or sulfuric acid as described in R. C. Larock “Comprehensive Organic Transformations” or for example in F. T. Schevenels, M. Shen. A. Scott “J. American Chemical Society”, 2017, vol.139 pages 6329-6337.

The compounds having formula (I) can also be obtained by Mitsunobu reaction between the acid having formula (II) and the alcohol having general formula (III) in the presence of triphenylphosphine and diethylazodicarboxylate, in a solvent such as, for example, tetrahydrofuran, diethyl ether or dioxane, at a temperature ranging from room temperature to the reflux temperature of the solvent, as described for example in U.S. Pat. No. 7,601,849 (2009).

Alternatively, the compounds having formula (I) can be obtained by activation of the carboxylic acid or via acyl chloride or via mixed anhydride and the subsequent addition of the appropriate alcohol having general formula (III), according to reaction scheme 2.

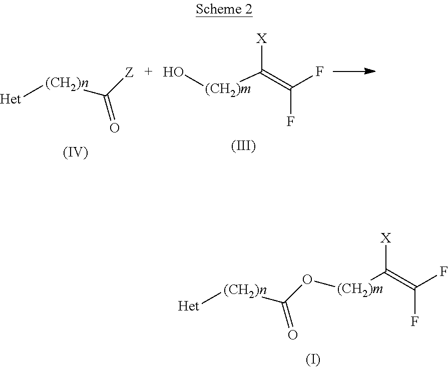

The reaction is carried out by reacting a compound having formula (IV), wherein Z represents a chlorine atom or an OCOR₁ residue, with R₁ which has the meaning of C₁-C₄ alkyl, obtained from the compound having general formula (II) by methods known in literature with an alcohol having general formula (III) in the presence of a base such as triethylamine or N-methyl-morpholine or pyridine, in a suitable solvent such as methylene chloride or chloroform or tetrahydrofuran, at a temperature ranging from 0° C. to the boiling point of the solvent, for a time ranging from 1 to 72 hours, as widely described in R. C. Larock “Comprehensive Organic Transformations” or for example in US 2003/109563 (2003) or in US 2004/198702 (2004).

The compounds having general formula (I) can also be obtained from a suitable salt of carboxylic acid having general formula (V), an alkaline metal salt, such as sodium, lithium or potassium or ammonium, such as trimethylammonium or triethylammonium, in the presence of a derivative having formula (VI) wherein K represents an outgoing group such as a halogen atom, selected from chlorine, bromine or iodine or a trifluoromethanesulfonate or p-toluenesulfonate, or methanesulfonate group according to reaction scheme 3.

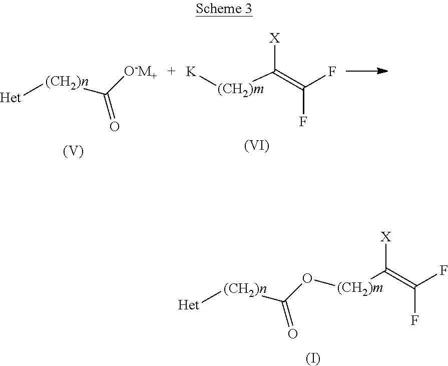

The reaction involves the salification of carboxylic acid with a base such as sodium bicarbonate, potassium bicarbonate, potassium carbonate, sodium carbonate, sodium hydroxide, potassium hydroxide, lithium hydroxide or sodium hydride or potassium t-butylate in a solvent such as tetrahydrofuran, N,N-dimethylformamide, N-methylpyrrolidone, toluene or acetone and the subsequent addition of a compound having formula (VI), at a temperature ranging from room temperature to the reflux temperature of the solvent selected, as described for example in U.S. Pat. No. 5,519,015 (1996).

The compounds having formula (II), when they are not commercial, can be easily prepared according to methods known in organic chemistry, as described in R. C. Larock “Comprehensive Organic Transformations”.

The compounds having formula (VI), with K which represents a halogen atom, are commercial products.

Alternatively, the compounds having formula (VI), with K which represents a trifluoromethanesulfonate, p-toluenesulfonate or methanesulfonate group, can be obtained from the corresponding alcohols (III) as described in Theodora W: Greene “Protective Groups in Organic Synthesis”. pages 198-199 and indicated in scheme 4.

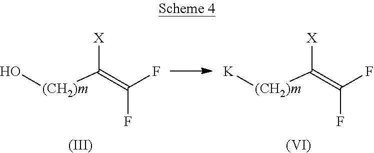

The compounds having general formula (I), for particular meanings of Het, can be obtained in racemic form or as optically active isomers.

Both the compounds having general formula (I) isomerically pure, and mixtures thereof, possibly obtained during the preparation of the compounds having general formula (I) or deriving from an incomplete separation of the same isomers, in any proportion, therefore fall within the scope of the present invention.

As already mentioned, the compounds having general formula (I) are provided with a high nematocidal activity and do not show any phytotoxicity with respect to the crops of application, making them suitable for use in the agrarian field in defense against nematodes.

A further object of the present invention therefore relates to the use of compounds having Formula (I) for the control of nematodes.

In particular, the compounds of the present invention are effective in the control of numerous nematodes such as, for example: Pratylenchus spp, Globodera spp, Heterodera spp, Meloidogyne spp, Aphelenchoides spp, Radopholus Similis, Ditylenchus Dipsaci, Tylenchulus Semipenetrans, Longidorus spp, Xiphinema spp, Trichodorus spp, Bursaphelenchus spp, Belonolaimus spp., etc.

More specifically, the compounds having Formula (I) can be applied at different times of the vegetative development, for example before the transplanting/sowing or during the growth of the plant, via the leaves, or to the soil by fertigation, or incorporation in the ground, or through seed tanning.

The compounds having Formula (I) are capable of exerting a curative and preventive nematocidal action and exhibit a very low or no phytotoxicity on the crops treated.

For practical uses in agriculture, it is often advantageous to use the compounds of the present invention appropriately formulated in agronomic compositions having a nematocidal activity comprising one or more compounds having Formula (I), possibly also as a mixture of agronomically acceptable isomers and coformulants.

A further object of the present invention therefore relates to nematocidal agronomic compositions comprising one or more compounds having Formula (I), a solvent and/or solid, liquid or liquefied diluent, optionally one or more surfactants and other agronomically acceptable coformulants.

Compositions can be used which are in the form of dry powders, wettable powders, emulsifiable concentrates, microemulsions, pastes, granulates, solutions, suspensions, fumigants etc.: the choice of the type of composition will depend on the specific use. The compositions are prepared according to known methods, for example by diluting or dissolving the active substance with a solid solvent and/or diluent, optionally in the presence of surfactants.

Kaolin, alumina, silica, talc, bentonite, gypsum, quartz, dolomite, attapulgite, montmorillonite, diatomaceous earth, cellulose, starch, etc. can be used as solid inert diluents, or carriers.

Liquid inert diluents that can be used are water, or organic solvents such as aromatic hydrocarbons (xylols, mixtures of alkylbenzenes, etc.), aliphatic hydrocarbons (hexane, cyclohexane, etc.), halogenated aromatic hydrocarbons (chlorobenzene, etc.), alcohols (methanol, propanol, butanol, octanol, etc.), esters (isobutyl acetate, etc.), ketones (acetone, cyclohexanone, acetophenone, isophorone, ethylamylketone, etc.), or vegetable or mineral oils or mixtures thereof, etc.

Propellant gases such as butane, propane, halogenated hydrocarbons, nitrogen or carbon dioxide can be used as liquefied diluents or liquefied substances that gasify at room temperature and pressure.

Surfactants that can be used are wetting agents and emulsifiers of the non-ionic type (polyethoxylated alkylphenols, polyethoxylated fatty alcohols, etc.), anionic type (alkylbenzene sulfonates, alkylsulfonates, etc.), cationic type (quaternary salts of alkylammonium, etc.).

Dispersants (e.g. lignin and its salts, cellulose derivatives, alginates, etc.), stabilizers (e.g. antioxidants, ultraviolet ray absorbents, etc.) can also be added.

The concentration of active substance in the above compositions can vary within a wide range, depending on the active compound, the applications for which they are intended, the environmental conditions and the type of formulation adopted. In general, the concentration of active substance preferably ranges from 0.1 to 90%, and in particular from 0.5 to 90%.

The compounds of the present invention, as such or formulated, can be used in a mixture with other active ingredients such as, for example, herbicides, fungicides, bactericides, insecticides, acaricides, nematocides, fertilizers, biostimulants, etc. to broaden the spectrum or prevent resistance.

In some cases, the mixtures thus obtained have a synergistic effect between the components, which brings the mixture, for example, to exert a higher activity with respect to that of the individual elements of which it is composed.

Examples of insecticides, acaricides, nematocides that can be added to the compositions containing one or more compounds having general formula (I) are the following: abamectin, acetamiprid, acrinathrin, alphacypermethrin, alphamethrin, azadirachtin, Bacillus subtilis, Bacillus thuringiensis, Beauveria bassiana, betacyfluthrin, bifenazate, bifenthrin, buprofezin, chlorpyrifos, chlorpyrifos M, clofentezine, cyhalothrin, cyhexatin, cypermethrin, cyromazine, chloropicrin, clorantranilipide, clotianidin, deltamethrin, diflubenzuron, dimethoat, dazonet, difluoruro di solforile, dimethyldisulfide, emamectin, esfenvalerate, ethoprophos, etofenprox, etoxazole, fenamiphos, fenazaquin, fenoxycarb, fenpyroximate, fipronil, fluazinam, fluensulfone, flufenoxuron, fluvalinate, fosthiazate, formentanate, flonicamid, formet, viruses, hexythiazox, imidaclopridi, indoxacarb, lambda-cyhalothrin, lufenuron malathion, metaldehyde, methamidophos, Metharhizium spp, methiocarb, methomyl, methoxyfenozide, milbemectin, metaflumizone, metam sodium, metam potassium, oxamyl, Paecilomyces fumosoroseus, phosmet, pirimicarb, pirimiphos M, pyrethrum, pyridaben, pyriproxyfen, piperonyl butoxide, spinosad, spironesifen, spirotetramat, spinetoran, spirodiclofen, tau-fluvalinate, tebufenozide, tebufenpyrad, teflubenzuron, tefluthrin, thiacloprid, triflumuron, zeta-cypermethrin, (1R-cis)-[5-(phenylmethyl)-3-furanyl]-methyl-3-[(dihydro-2-oxo-3(2H)-furanylidene) methyl]-2,2-dimethylcyclo-propanecarboxylate, (3-phenoxyphenyl)-methyl-2,2,3 ,3 -tetramethyl-cyclopropane-carboxylate, 1-[(2-chloro-5-thiazolyl)methyl]-5-triazine 2-(1H)-imine, 2-(2-chloro-6-fluorophenyl)-4-[4-(1,1-dimethylethyl)phenyl]-4,5-dihydro-oxazole, 2-(acetyloxy)-3-dodecyl-1,4-naphthalenedione, 2-chloro-N-[[[4-(1-phenyleneoxy)-phenyl]-amino]-carbonyl]-benzamide, 2-chloro-N-[[[4-(2,2-dichloro-1,1-difluoroethoxy)-phenyl]amino]carbonyl]-benzamide, 3-methylphenyl-propylcarbamate, 4-[4-(4-ethoxyphenyl)-4-methylpentyl]-1-fluoro-2-phenoxybenzene, 4-chloro-2-(1,1-dimethylethyl)-5-[[2-(2,6-dimethyl-4-phenoxyphenoxy)ethyl]thio]-3 -(2H)-pyridazinone, 4-chloro-2-(2-chloro-2-methylpropyl)-5-[(6-iodo-3-pyridinyl)(2-chloro-2-methylpropyl)-5-[(6-iodo-3-pyridinyl)-methoxy]-3 -(2H)pyridazinone, 4-chloro-5-[(6-chloro-3-pyridinyl) methoxy]-2-(3,4-dichlorophenyl)-3(2H)-pyridazinone, Bacillus thuringiensis strain EG-2348, [2-benzoyl-1-(1,1-dimethylethyl)-hydrazine]benzoic acid, 2,2-dimethyl-3-(2,4-dichloro-phenyl)-2-oxo-1-oxaspire[4.5]-dec-3-ene-4-butanoate, [3-[(6-chloro-3-pyridinyl)-methyl]-2-thiazolidinylidene]-cyanamide, dihydro-2-(nitromethylene)-2H-1,3-thiazine-3(4H)-carboxymaldehyde, ethyl [2-[[1,6-dihydro-6-oxo-1-(phenylmethyl)-4-pyridazinyl]oxy]ethyl]-carbamate, N-(3 ,4,4-trifluoro-1-oxo-3-butenyl)-glycine, N-(4-chlorophenyl)-3-[4-(difluoromethoxy)-phenyl]-4,5-dihydro-4-phenyl-1H-pyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl[2-(dipropylamino)-2-oxoethyl]-ethyl-phosphoramidothioate.

Examples of herbicides that can be added to the compositions containing one or more compounds having general formula (I) are the following:

acetochlor, acifluorfen, aclonifen, AKH-7088 ({methyl (E,Z)-[[[1-[2-chloro-4-(trifluoromethyl)phenoxy]-2-nitrophenyl]-2-methoxyethylidene]amino]acetate}), alachlor, alloxydim, ametryn, amicarbazone, amidosulfuron, amitrole, anilofos, asulam, atrazine, azafenidin, azimsulfuron, aziprotryne, BAY MKH 6561 (sodium salt of methyl 2-({[(4-methyl-5-oxo-3-propoxy-4,5-dihydro-1H-1,2,4-triazol-1-yl)carbonyl]amino}-sulfonyl)benzoate), beflubutamid, benazolin, benfluralin, benfuresate, bensulfuron, bensulide, bentazone, benzfendizone, benzobicyclon, benzofenap, benzthiazuron, bifenox, bilanafos, bispyribac-sodium, bromacil, bromobutide, bromofenoxim, bromoxynil, butachlor, butafenacil, butamifos, butenachlor, butralin, butroxydim, butylate, cafenstrole, carbetamide, carfentrazone-ethyl, chlomethoxyfen, chloramben, chlorbromuron, chlorbufam, chlorflurenol, chloridazon, chlorimuron, chlornitrofen, chlorotoluron, chloroxuron, chlorpropham, chlorsulfuron, chlorthal, chlorthiamid, cinidon ethyl, cinmethylin, cinosulfuron, clethodim, clodinafop, clomazone, clomeprop, clopyralid, cloransulam-methyl, cumyluron (JC-940), cyanazine, cycloate, cyclosulfamuron, cycloxydim, cyhalofop-butyl, 2,4-D, 2,4-DB, daimuron, dalapon, desmedipham, desmetryn, dicamba, dichlobenil, dichlorprop, dichlorprop-P, diclofop, diclosulam, diethatyl, difenoxuron, difenzoquat, diflufenican, diflufenzopyr, dimefuron, dimepiperate, dimethachlor, dimethametryn, dimethenamid, dinitramine, dinoseb, dinoseb acetate, dinoterb, diphenamid, dipropetryn, diquat, dithiopyr, 1-diuron, eglinazine, endothal, EPTC, esprocarb, ethalfluralin, ethametsulfuron-methyl, ethidimuron, ethiozin (SMY 1500), ethofumesate, ethoxyfen-ethyl (HC-252), ethoxysulfuron, etobenzanid (HW 52), fenoxaprop, fenoxaprop-P, fentrazamide, fenuron, flamprop, flamprop-M, flazasulfuron, florasulam, fluazifop, fluazifop-P, fluazolate (JV 485), flucarbazone-sodium, fluchloralin, flufenacet, flufenpyr ethyl, flumetsulam, flumiclorac-pentyl, flumioxazin, flumipropin, fluometuron, fluoroglycofen, fluoronitrofen, flupoxam, flupropanate, flupyrsulfuron, flurenol, fluridone, flurochloridone, fluroxypyr, flurtamone, fluthiacet-methyl, fomesafen, foramsulfuron, fosamine, furyloxyfen, glufosinate, glyphosate, halo sulfuron-methyl, haloxyfop, haloxyfop-P-methyl, hexazinone, imazamethabenz, imazamox, imazapic, imazapyr, imazaquin, imazethapyr, imazosulfuron, indanofan, iodosulfuron, ioxynil, isopropalin, isoproturon, isouron, isoxaben, isoxachlortole, isoxaflutole, isoxapyrifop, KPP-421, lactofen, lenacil, linuron, LS830556 ([[[2-methyl(methylsulfonyl)amino]-2-oxoethyl]amino] methyl-phosphonic acid), MCPA (2-methyl-4-chlorophenoxyacetic acid), MCPA-thioethyl, MCPB (4-(4-chloro-2-methylphenoxy)butanoic acid), mecoprop, mecoprop-P, mefenacet, mesosulfuron, mesotrione, metamitron, metazachlor, methabenzthiazuron, methazole, methoprotryne, methyldymron, metobenzuron, metobromuron, metolachlor, S-metolachlor, metosulam, metoxuron, metribuzin, metsulfuron, molinate, monalide, monolinuron, naproanilide, napropamide, naptalam, NC-330 (methyl 5-[(4,6-dimethylpyrimidin-2-yl)carbamoylsulfamoyl]1-pyridin-2-yl pyrazole-4-carboxylate), neburon, nicosulfuron, nipyraclofen, norflurazon, orbencarb, oryzalin, oxadiargyl, oxadiazon, oxasulfuron, oxaziclomefone, oxyfluorfen, paraquat, pebulate, pendimethalin, penoxsulam, pentanochlor, pentoxazone, pethoxamid, phenmedipham, picloram, picolinafen, piperophos, pretilachlor, primisulfuron, prodiamine, profluazol, proglinazine, prometon, prometryne, propachlor, propanil, propaquizafop, propazine, propham, propisochlor, propyzamide, prosulfocarb, prosulfuron, pyraclonil, pyraflufen-ethyl, pyrazogyl (HSA-961), pyrazolynate, pyrazosulfuron, pyrazoxyfen, pyribenzoxim, pyributicarb, pyridafol, pyridate, pyriftalid, pyriminobac-methyl, pyrithiobac-sodium, pyroxasulfone quinclorac, quinmerac, quizalofop, quizalofop-P, rimsulfuron, sethoxydim, siduron, simazine, simetryn, sulcotrione, sulfentrazone, sulfometuron-methyl, sulfosulfuron, 2,3,6-TBA, TCA-sodium, tebutam, tebuthiuron, tepraloxydim, terbacil, terbumeton, terbuthyl-azine, terbutryn, thenylchlor, thiazafluron, thiazopyr, thidiazimin, thifensulfuron-methyl, thiobencarb, tiocarbazil, tioclorim, tralkoxydim, tri-allate, triasulfuron, triaziflam, tribenuron, triclopyr, trietazine, trifloxysulfuron, trifluralin, triflusulfuron-methyl, trito sulfuron, UBI-C4874 (quizalofop-P), vernolate.

Examples of fungicides that can be added to the compositions containing one or more compounds having general formula (I) are the following:

acibenzolar, ametoctradin, amisulbrom, ampropylfos, anilazine, azaconazole, azoxystrobin, benalaxyl, benalaxyl-M, benomyl, benthiavalicarb, bitertanol, bixafen, blasticidin-S, boscalid, bromuconazole, bupirimate, buthiobate, captafol, captan, carbendazim, carboxin, carpropamid, chinomethionat, chloroneb, chlorothalonil, chlozolinate, cyazofamid, cyflufenamid, cymoxanil, cyproconazole, cyprodinil, debacarb, dichlofluanid, dichlone, diclobutrazol, diclomezine, dicloran, diclocymet, diethofencarb, difenoconazole, diflumetorim, dimethirimol, dimethomorph, dimoxystrobin, diniconazole, dinocap, dipyrithione, ditalimfos, dithianon, dodemorph, dodine, edifenphos, epoxiconazole, etaconazole, ethaboxam, ethirimol, ethoxyquin, etridiazole, famoxadone, fenamidone, fenaminosulf, fenapanil, fenarimol, fenbuconazole, fenfuram, fenhexamid, fenoxanil, fenpiclonil, fenpropidin, fenpropimorph, fenpyrazamine, fentin, ferbam, ferimzone, fluazinam, fludioxonil, fluindapyr, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpet, fosetyl-aluminium, fuberidazole, furalaxyl, furametpyr, furconazole, furconazole-cis, guazatine, hexaconazole, hymexazol, idrossichinolina solfato, imazalil, imibenconazole, iminoctadine, ipconazole, iprobenfos, iprodione, isoprothiolane, iprovalicarb, isopyrazam, isotianil, kasugamycin, kresoxim-methyl, mancopper, mancozeb, mandipropamid, maneb, mebenil, mepanipyrim, mepronil, meptyldinocap, metalaxyl, metalaxyl-M, metconazole, methfuroxam, metiram, metominostrobin, metrafenone, metsulfovax, myclobutanil, natamycin, nicobifen, nitrothal-isopropyl, nuarimol, ofurace, orysastrobin, oxadixyl, oxpoconazole, oxycarboxin, pefurazoate, penconazole, pencycuron, penflufen, pentachlorofenol and its salts, penthiopyrad, phthalide, picoxystrobin, piperalin, Bordeaux mixture, polyoxins, probenazole, prochloraz, procymidone, propamocarb, propiconazole, propineb, proquinazid, prothiocarb, prothioconazole, pyracarbolid, pyraclostrobin, pyrametostrobin, pyraoxystrobin, pyrazophos, pyribencarb, pyrifenox, pyrimethanil, pyriofenone, pyroquilon, pyroxyfur, quinacetol, quinazamid, quinconazole, quinoxyfen, quintozene, rabenzazole, copper hydroxide, copper oxychloride, copper (I) oxide, copper sulfate, sedaxane, silthiofam, simeconazole, spiroxamine, streptomycin, tebuconazole, tebufloquin, tetraconazole, thiabendazole, thiadifluor, thicyofen, thifluzamide, thiophanate, thiophanate-methyl, thiram, tiadinil, tioxymid, tolclofos-methyl, tolylfluanid, triadimefon, triadimenol, triarimol, triazbutil, triazoxide, tricyclazole, tridemorf, trifloxystrobin, triflumizole, triforine, triticonazole, uniconazole, uniconazole-P, validamycin, valifenalate, vinclozolin, zineb, ziram, sulfur, zoxamide.

Examples of bactericides that can be added to the compositions containing one or more compounds having general formula (I) are the following:

bronopol, dichlorophen, nitrapyrin, nickel dimethyldithiocarbamate, kasugamycin, octhylinone, furancar-boxylic acid, probenazole, streptomycin, tecloftalam, copper hydroxide, copper oxychloride, copper (I) oxide, copper sulfate, copper salicylate.

Examples of fertilizers and biostimulants that can be added to the compositions containing one or more compounds having general formula (I) are the following:

mixtures of amino acids and/or oligopeptides of an animal and/or vegetable origin, 4-thiazolidanecarboxylic acid, 4-acetylthiazolidine-carboxylic acid, ectoin, phytosterols.

A further object of the present invention therefore relates to agronomic compositions comprising at least one compound having Formula (I) and at least one second active ingredient selected from insecticides, acaricides, nematocides different from those having Formula (I), herbicides, fungicides, bactericides, fertilizers and biostimulants.

The compositions, object of the present invention, are capable of exerting a nematocidal action which can be of a curative and/or preventive nature and, in general, exhibit very low or no phytotoxicity with respect to the crops treated.

A further object of the present invention therefore relates to the use of the compositions comprising at least one compound having Formula (I) for the control of nematodes.

If the compositions comprise a compound having Formula (I) and at least one further known active ingredient, the weight ratios in the above compositions, between the compound having Formula (I) and the other known active ingredients, vary according to the compounds selected and can normally be within the range of 1:100 to 100:1, preferably from 1:10 to 10:1.

The total concentration of the active components in the above compositions can vary within a wide range; it generally ranges from 1% to 99% by weight with respect to the total weight of the composition, preferably from 5% to 90% by weight with respect to the total weight of the composition.

The compounds having Formula (I) or the compositions containing them can be applied to the crop via the leaves, or to the soil by fertigation, or incorporation in the ground, or through seed tanning.

A further object of the present invention therefore relates to a method for controlling nematodes in cultivated areas, which consists in applying effective and non-phytotoxic doses of compositions comprising at least one compound having Formula (I) and, optionally, one or more known active ingredients compatible therewith, on any part of the plant to be protected.

The quantity of compound to be applied for obtaining the desired effect can vary depending on various factors such as, for example, the compound used, the crop to be protected, the degree of infestation, the climatic conditions, the characteristics of the soil, the method of application, etc.

Doses of compound having Formula (I) ranging from 100 g to 10,000 g per hectare of agricultural crop or, in the case of compositions comprising other known active ingredients, overall doses of active ingredients ranging from 100 g to 20,000 g per hectare of agricultural crop, generally provide a sufficient control.

Doses of compound having Formula (I) ranging from 500 g to 800 g per hectare of agricultural crop are preferably used.

The following examples are now provided for a better illustration of the present invention, which should be considered illustrative and non-limiting thereof.

### EXAMPLE 1

### Preparation of 5-methyl-1,3,4-thiadiazole-2-carboxylate of 3,4,4-trifluoro-3-butenyl [Compound Nr. 4]

a) Preparation of the Lithium Salt of 5-methyl-1,3,4-thiadiazole-2-carboxylic acid Having General Formula (II)

4 ml of an aqueous solution containing 0.047 g (1.97 mmoles) of lithium hydroxide at 0° C. was added under stirring to a solution of 0.340 g (1.97 mmoles) of ethyl 5-methyl-1,3,4-thiadiazole-2-carboxylate in 4 ml of tetrahydrofuran. The reaction mixture was then brought to room temperature and left under stirring for one night.

The following day the solvent was evaporated and the lithium salt of 5-methyl-1,3,4-thiadiazole-2-carboxylic acid was used as such for the subsequent reaction.

b) Preparation of 5-methyl-1,3,4-thiadiazole-2-carboxylate of 3,4,4-trifluoro-3-butenyl [Compound Nr. 4].

0.84 g (4.46 mmoles) of 1-bromo-3,4,4-trifluoro-3-butene were added to a solution of 0.28 g (1.94 mmoles) of lithium salt of 5-methyl-1,3,4-thiadiazole-2-carboxylic acid, obtained from the previous reaction, dissolved in 6 ml of N,N -dimethylformamide. The mixture was left under magnetic stirring at room temperature for three hours.

After control in LC-MS, the mixture was diluted with water and the phases were then separated; the aqueous phase was re-extracted twice with ethyl acetate. The combined organic phases were thoroughly washed with a saturated solution of sodium chloride and subsequently with water. After drying on sodium sulfate, filtration and evaporation of the solvent at reduced pressure, 0.31 g of orange oil (1.3 mmoles) were obtained. The raw product thus obtained was purified by chromatography on silica gel eluting with a mixture of heptane:ethyl acetate 3:2, obtaining 0.29 g (1.15 mmoles) of the desired product.

Yield 60%

LC-MS [M+H]=253

### EXAMPLE 2

### Preparation of 2-methyl-4-difluoromethyl-thiazole-5-carboxylate of 3,4,4-trifluoro-3-butenyl [Compound Nr. 2]

1 g (5.17 mmoles) of 2-methyl-4-difluoromethyl-thiazole-5-carboxylic acid was dissolved in a nitrogen atmosphere in 20 ml of N,N-dimethylformamide. 1.1 g (7.75 mmoles) of potassium carbonate and 2.25 g of 1-bromo-3,4,4-trifluoro-3-butene were then added, under stirring and maintaining the reaction mixture at room temperature. After the addition, the mixture was heated to 70° C. for 3 hours and left at room temperature for a night.

After control in LC-MS, the mixture was diluted with water and the phases were then separated; the aqueous phase was re-extracted twice with ethyl acetate. The combined organic phases were washed thoroughly with a saturated sodium chloride solution and subsequently with water. After drying on sodium sulfate, filtration and evaporation of the solvent at reduced pressure, 1.42 g of yellow oil (4.7 mmoles) were obtained.

Yield 92%

LC-MS [M+H]=302.

### EXAMPLE 3

Operating analogously to what is indicated in Examples 1 and 2, Compounds 1, 3, 8 12, 13, 14, 15, 16, 17, 118, 122, 123, 124, 125, 126, 127, 128, 129, 131, 132 were prepared, shown in Table 3.

TABLE 3

| Comp. Nr. | Het | n | m | X |
|---|---|---|---|---|
| 1 | 5-Cl-thiazol-2-yl | 0 | 2 | F |
| 3 | 2-CH₃-4-CF₃-thiazol-5-yl | 0 | 2 | F |
| 8 | 2-CF₃-thiazol-4-yl | 0 | 2 | F |
| 12 | 2-CF₃-pyridyl-3-yl | 0 | 2 | F |
| 13 | 1-CH₃-3-CF₂H-pyrazol-4-yl | 0 | 2 | F |
| 14 | 2-Cl-furan-3-yl | 0 | 2 | F |
| 15 | 5-iPr-1,3,4-thiadiazol-2-yl | 0 | 2 | F |
| 16 | 1-CH₃-2-CH₃-5-CH₃-pyrazol-4-yl | 0 | 2 | F |
| 17 | 6-CF₃-8-Cl-imidazo-[1,2-a]-pyridyl-2-yl | 0 | 2 | F |
| 118 | 1-CH₃-3-CF₃-pyrazol-4-yl | 0 | 2 | F |
| 122 | 2-Ph-4-CH₃-1,3-thiazol-5-yl | 0 | 2 | F |
| 123 | 6-CF₃-pyridin-3-yl | 0 | 2 | F |
| 124 | 2-CF₃-6-Cl-pyridin-3-yl | 0 | 2 | F |
| 125 | 4-CF₃-pyridin-3-yl | 0 | 2 | F |
| 126 | Pyrimidin-2-yl | 0 | 2 | F |
| 127 | Pyrimidin-5-yl | 0 | 2 | F |
| 128 | Pyrimidin-4-yl | 0 | 2 | F |
| 129 | 5-Me-1,2-oxazol-3-yl | 0 | 2 | F |
| 131 | Pyridin-3-yl | 2 | 2 | F |
| 132 | Pyridin-3-yl | 1 | 2 | F |

Table 4 shows the results of the LC-MS analyses conducted on Compounds 1, 3, 8, 12, 13, 14, 15, 16, 17, 118, 122, 123, 124, 125, 126, 127, 128, 129, 131, 132.

TABLE 4

| Comp. Nr. | LC-MS: [M + H]+ |
|---|---|
| 1 | 272 |
| 3 | 320 |
| 8 | 306 |
| 12 | 300 |
| 13 | 285 |
| 14 | 253 |
| 15 | 281 |
| 16 | 263 |
| 17 | 373 |
| 118 | 303 |
| 122 | 328 |
| 123 | 300 |
| 124 | 334 |
| 125 | 300 |
| 126 | 233 |
| 127 | 233 |
| 128 | 233 |
| 129 | 236 |
| 131 | 260 |
| 132 | 246 |

### EXAMPLE 4

Determination of Nematocidal Activity Against Meloidogyne sp.

The tests aimed at testing the nematocidal activity of the product under examination were carried out using inoculae taken from a farming of Meloidogyne sp. maintained on potted tomato and cucumber plants and grown in greenhouses.

To carry out the experiments, portions of roots having a good number of galls and soil were taken from the infested pots, in which larvae were present starting from the second stage of age.

New pots having a diameter of 15 cm were half filled with sterile soil. The portions of infested roots, previously cleaned up, were placed on the same in order to be able to correctly assess the degree of infestation and ensure that each pot contains the same nematic charge. 200-300 g of infested soil were subsequently added, then covered with a thin layer of sterile soil.

The treatment was carried out by pouring 100 ml of solution on the surface of the soil, in which the product to be tested was dissolved.

Tomato or cucumber seedlings at the stage of two or three true leaves were transplanted in the pots thus prepared, one or seven days after application. Different cultivars of tomato or cucumber were used, having a different sensitivity to the parasite and different growth times. In particular, a variety of ornamental tomatoes (cv Microtom) was used for assessing the final production, whose seedlings are small in size and are able to reach the ripeness of the fruit in pots and under greenhouse conditions in about two months

The product was evaluated considering the possible phytotoxic effects that may compromise the development of the plant (in this case a zero-ten scale was used, according to which zero=no symptoms, 10=destroyed plant) and the effectiveness against nematodes.

The containment capacity of the parasite was detected, 30 and 60 days after transplantation, considering the development of the root system (where 100% is the development achieved by the healthy comparative root) and the presence of galls on the roots. This was estimated using the infestation scale proposed by Bridge-Page, according to which the value zero corresponds to 0% of the root affected and the value 10 corresponds to 100% of infested root.

Table 5 shows the results relating to the phytotoxicity on cucumbers, cv Chinese Long, treating with Compounds Nr. 2, 4, 12, 13, 17 included in general formula (I) and CR1, described in JP 2000038379 instead of the compound having general formula (I).

TABLE 5

| | Phytotoxicity |
|---|---|
| Comp. Nr. | (scale 0-10) |
| 2 | 0 |
| 4 | 0 |
| 12 | 0 |
| 13 | 0 |
| 17 | 0 |
| CR1 | 0 |

CR1 = pyridyl-3-carboxylate of 3,4,4-trifluoro-3-butenyl

Table 6 shows the results relating to the effectiveness of Compounds Nr. 2, 4, 12, 13, 17 and the Compound CR1, on cucumbers, cv Chinese Long, at a dose of 4,000 g/hectare, effecting the survey 30 days after transplantation.

TABLE 6

| | | % | ROOT |
|---|---|---|---|
| | ALL INDEX | INFESTED | DEVELOP- |
| COMPOUND | scale 0-10) | ROOT | MENT |
| INFESTED | 2.5 | 30 | 40 |
| BLANK | | | |
| Comp. Nr. 2 | 0.3 | 3 | 90 |
| Comp. Nr. 4 | 0.3 | 3 | 90 |
| Comp. Nr. 12 | 0 | 0 | 95 |
| Comp. Nr. 13 | 0 | 0 | 95 |
| Comp. Nr. 17 | 0.3 | 3 | 85 |
| CR1 | 0.8 | 8 | 70 |

CR1 = pyridyl-3-carboxylate of 3,4,4-trifluoro-3-butenyl

Table 7 shows the results relating to the effectiveness of Compounds Nr. 2, 4, 8, 12, 13, 17, 127, 129 and the Compound CR1, on cucumbers, cv Chinese Long, at doses of 2,000 g/hectare, 1,000 g/hectare and 500 g/hectare effecting the survey 30 days after transplantation.

TABLE 7

| | |
|---|---|
| GALL INDEX | WEIGHT OF |
| (scale 0-10) | FRESH PLANT (g) |

DOSE APPLIED (kg ai/ha)

| COMPOUND | 2 | 1 | 0.5 | 2 | 1 | 0.5 |
|---|---|---|---|---|---|---|
| HEALTHY | / | | | 37 | | |
| BLANK | | | | | | |
| INFESTED | 7 | | | 16 | | |
| BLANK | | | | | | |
| Comp. Nr. 2 | 1.4 | 2.6 | 2.8 | 35 | 37 | 36 |
| Comp. Nr. 4 | 1.9 | 1.9 | 2.2 | 34 | 37 | 35 |
| Comp. Nr. 8 | 1.2 | 2.3 | 2.5 | 37 | 27 | 25 |
| Comp. Nr. 12 | 2.1 | 2.9 | 3.1 | 31 | 28 | 27 |
| Comp. Nr. 13 | 1.2 | 1.2 | 2.1 | 39 | 38 | 36 |
| Comp. Nr. 17 | 2.2 | 2.8 | 3.3 | 31 | 28 | 26 |
| Comp. Nr. 127 | 0.9 | 2.1 | 3.4 | 36 | 29 | 26 |
| Comp. Nr. 129 | 0.9 | 2.4 | 3.6 | 35 | 26 | 25 |
| CR1 | 2.8 | 3.5 | 4.8 | 30 | 25 | 23 |

CR1 = pyridyl-3-carboxylate of 3,4,4-trifluoro-3-butenyl

As can be seen in Table 7, the compounds according to the invention are more effective than the reference compound CR1 at all the doses tested, up to 500 g of active principle per hectare.

Furthermore, the application of the products of the present invention leads to a fresh weight of the plant higher than that obtained with the application of the compound CR1, thus demonstrating the excellent health of the plant treated.

1. An ester of heterocyclic compounds having a general formula 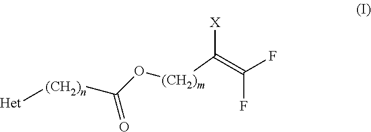
wherein:
Het represents an aromatic heterocycle having from 5 to 10 terms, possibly benzofused, or heterobicyclic containing heteroatoms selected from nitrogen, sulfur, and oxygen; said heterocycle being optionally substituted by one or more Y groups selected from halogen, C₁-C₆-alkyl, C₁-C₆-haloalkyl, C₃-C₆-cycloalkyl, C₄-C₇-cycloalkylalkyl, C₁-C₆-alkoxyl, C₁-C₆-haloalkoxyl, C₁-C₆-thioalkoxyl, C₁-C₆-thiohaloalkoxyls, C₁-C₆-alkylsulfinyl, C₁-C₆-alkylsulfonyl, C₁-C₆-alkoxycarbonyl, C₃-C₆-cycloalkoxycarbonyl, amino, N—C₁-C₆-alkylamino, N,N—C₂-C₁₂-dialkylamino, N—C₁-C₆-alkoxycarbonylamino, N—C₃-C₆-cycloalkylamino, N,N—C₆-C₁₂-dicycloalkylamino, N—C₃-C₆-cycloalkoxycarbonylamino, C₁-C₆-alkylaminocarbonyl, C₃-C₆-cycloalkylaminocarbonyl, a NR₁R₂CONR₁-formyl group, C₁-C₆-alkylcarbonyl, carboxyl, cyano, an aryl, optionally substituted, a benzyl, an aromatic heterocycle, optionally substituted, penta- or hexa-atomic also benzo-fused or heterobicyclic, containing at least one heteroatom selected from oxygen, sulfur, nitrogen, possibly oxidized to N-oxide;
n represents an integer ranging from 0 to 4;
m represents an integer ranging from 1 to 6;
X represents an H or fluorine atom;
R₁, R₂ the same or different, represent a hydrogen atom, a C₁-C₄ alkyl group or a C₃-C₆-cycloalkyl group,
wherein
if n has the value of 1, X represents an F atom for any meaning of Het and m;
if Het represents a thiophen-2-yl group or a furan-2-yl group or a pyrazin-2-yl group or a quinol-4-yl or pyrrolyl-2-yl group, substituted or not substituted by one or more Y groups or Het represents a pyrazol-5-yl group or a pyridyl-2-yl or pyridyl-3-yl or pyridyl-4-yl group not substituted or substituted by one or more Y groups selected from halogen, C₁-C₆-alkyl, C₁-C₆-alkoxyl, C₁-C₆-thioalkoxyl, C₁-C₆-thiohaloalkoxyls, aryl, or Het represents a 1,2,3-thiadiazol-4-yl group or a 1,2,3-thiadiazol-5-yl group substituted or not substituted by one or more Y groups, then, contemporaneously, n must be different from 0 and X different from H;
if Het represents a pyrazol-4-yl group substituted or not substituted by one or more Y groups, then contemporaneously n must be different from 1, m different from 1 and X different from F.

2. The ester according to claim 1, wherein Het, n, m and X in formula (I) have the meanings indicated in the following Table

| Comp. Nr. | Het | n | m | X |
|---|---|---|---|---|
| 1 | 5-Cl-thiazol-2-yl | 0 | 2 | F |
| 2 | 2-CH₃-4-CF₂H-thiazol-5-yl | 0 | 2 | F |
| 3 | 2-CH₃-4-CF₃-thiazol-5-yl | 0 | 2 | F |
| 4 | 5-CH₃-1,3,4-thiadiazol-2-yl | 0 | 2 | F |
| 5 | 5-CH₃-1,3,4-oxadiazol-2-yl | 0 | 2 | F |
| 6 | 5-iPr-1,3-oxazol-4-yl | 0 | 2 | F |
| 7 | 2-CH₃-5-CF₃-oxazol-4-yl | 0 | 2 | F |
| 8 | 2-CF₃-thiazol-4-yl | 0 | 2 | F |
| 9 | 2-iPr-thiazol-4-yl | 0 | 2 | F |
| 10 | 2-iPr-4-CH₃-1,3-thiazol-5-yl | 0 | 2 | F |
| 11 | Oxazol-5-yl | 0 | 2 | F |
| 12 | 2-CF₃-pyridyl-3-yl | 0 | 2 | F |
| 13 | 1-CH₃-3-CF₂H-pyrazol-4-yl | 0 | 2 | F |
| 14 | 2-Cl-furan-3-yl | 0 | 2 | F |
| 15 | 5-iPr-1,3,4-thiadiazol-2-yl | 0 | 2 | F |
| 16 | 1-CH₃-2-CH₃-5-CH₃-pyrazol-4-yl | 0 | 2 | F |
| 17 | 6-CF₃-8-Cl-imidazo-[1,2-a]-pyridyl-2-yl | 0 | 2 | F |
| 18 | Benzothiazol-2-yl | 0 | 2 | F |
| 19 | 5-CH₃-benzothiazol-2-yl | 0 | 2 | F |
| 20 | Benzothien-2-yl | 0 | 2 | F |
| 21 | Benzofuran-2-yl | 0 | 2 | F |
| 22 | 5-CH₃-[1,2,4] -triazole- [3,2-b]-thiazol-6-yl | 1 | 2 | F |
| 23 | 2-CH₃-thiazol-5-yl | 1 | 2 | F |
| 24 | 2-CH₃-4-CH₃-thiazol-5-yl | 1 | 2 | F |
| 25 | 2-CH₃-4-CF₂H-thiazol-5-yl | 0 | 4 | F |
| 26 | 2-CH₃-4-CF₃-thiazol-5-yl | 0 | 4 | F |
| 27 | 5-CH₃-1,3,4-thiadiazol-2-yl | 0 | 4 | F |
| 28 | 5-CH₃-1,3,4-oxadiazol-2-yl | 0 | 4 | F |
| 29 | 5-iPr-1,3-oxazol-4-yl | 0 | 4 | F |
| 30 | 2-CH₃-5-CF₃-oxazol-4-yl | 0 | 4 | F |
| 31 | 2-CF₃-thiazol-4-yl | 0 | 4 | F |
| 32 | 2-iPr-thiazol-4-yl | 0 | 4 | F |
| 33 | 2-iPr-4-CH₃-1,3-thiazol-5-yl | 0 | 4 | F |
| 34 | Oxazol-5-yl | 0 | 4 | F |
| 35 | 2-CF₃-pyridyl-3-yl | 0 | 4 | F |
| 36 | 1-CH₃-3-CF₂H-pyrazol-4-yl | 0 | 4 | F |
| 37 | 2-Cl-furan-3-yl | 0 | 4 | F |
| 38 | 5-iPr-1,3,4-thiadiazol-2-yl | 0 | 4 | F |
| 39 | 1-CH₃-2-CH₃-5-CH₃-pyrazol-4-yl | 0 | 4 | F |
| 40 | 6-CF₃-8-Cl-imidazo-[1,2-a]-pyridyl-2-yl | 0 | 4 | F |
| 41 | Benzothiazol-2-yl | 0 | 4 | F |
| 42 | 5-CH₃-benzothiazol-2-yl | 0 | 4 | F |
| 43 | Benzothien-2-yl | 0 | 4 | F |
| 44 | Benzofuran-2-yl | 0 | 4 | F |
| 45 | 5-CH₃-[1,2,4]-triazole-[3,2-b]-thiazol-6-yl | 1 | 4 | F |
| 46 | 2-CH₃-thiazol-5-yl | 1 | 4 | F |
| 47 | 2-CH₃-4-CH₃-thiazol-5-yl | 1 | 4 | F |
| 48 | 2-CH₃-4-CF₂H-thiazol-5-yl | 0 | 4 | F |
| 49 | 2-CH₃-4-CF₃-thiazol-5-yl | 0 | 4 | F |
| 50 | 5-CH₃-1,3,4-thiadiazol-2-yl | 0 | 4 | F |
| 51 | 5-CH₃-1,3,4-oxadiazol-2-yl | 0 | 4 | F |
| 52 | 2-CF₃-pyridyl-3-yl | 0 | 4 | F |
| 53 | 5-iPr-1,3-oxazol-4-yl | 0 | 4 | F |
| 54 | 2-CH₃-5-CF₃-oxazol-4-yl | 0 | 4 | F |
| 55 | 2-CF₃-thiazol-4-yl | 0 | 4 | F |
| 56 | 2-iPr-thiazol-4-yl | 0 | 4 | F |
| 57 | 2-iPr-4-CH₃-1,3-thiazol-5-yl | 0 | 4 | F |
| 58 | Oxazol-5-yl | 0 | 4 | F |
| 59 | 1-CH₃-3-CF₃-pyrazol-4-yl | 0 | 4 | F |
| 60 | 2-Cl-furan-3-yl | 0 | 4 | F |
| 61 | 5-iPr-1,3,4-thiadiazol-2-yl | 0 | 4 | F |
| 62 | 1-CH₃-2-CH₃-5-CH₃-pyrazol-4-yl | 0 | 4 | F |
| 63 | 6-CF₃-8-Cl-imidazo-[1,2-a]-pyridyl-2-yl | 0 | 4 | F |
| 64 | Benzothiazol-2-yl | 0 | 4 | F |
| 65 | 5-CH₃-benzothiazol-2-yl | 0 | 4 | F |
| 66 | Benzothien-2-yl | 0 | 4 | F |
| 67 | Benzofuran-2-yl | 0 | 4 | F |
| 68 | 5-CH₃-[1,2,4]-triazole-[3,2-b]-thiazol-6-yl | 1 | 4 | F |
| 69 | 2-CH₃-thiazol-5-yl | 1 | 4 | F |
| 70 | 2-CH₃-4-CH₃-thiazol-5-yl | 1 | 4 | F |
| 71 | 2-CH₃-4-CF₂H-thiazol-5-yl | 0 | 2 | H |
| 72 | 2-CH₃-4-CF₃-thiazol-5-yl | 0 | 2 | H |
| 73 | 5-CH₃-1,3,4-thiadiazol-2-yl | 0 | 2 | H |
| 74 | 5-CH₃-1,3,4-oxadiazol-2-yl | 0 | 2 | H |
| 75 | 5-iPr-1,3-oxazol-4-yl | 0 | 2 | H |
| 76 | 2-CH₃-5-CF₃-oxazol-4-yl | 0 | 2 | H |
| 77 | 2-CF₃-thiazol-4-yl | 0 | 2 | H |
| 78 | 2-iPr-thiazol-4-yl | 0 | 2 | H |
| 79 | 2-iPr-4-CH₃-1,3-thiazol-5-yl | 0 | 2 | H |
| 80 | Oxazol-5-yl | 0 | 2 | H |
| 81 | 2-CF₃-pyridyl-3-yl | 0 | 2 | H |
| 82 | 1-CH₃-3-CF₂H-pyrazol-4-yl | 0 | 2 | H |
| 83 | 2-Cl-furan-3-yl | 0 | 2 | H |
| 84 | 5-iPr-1,3,4-thiadiazol-2-yl | 0 | 2 | H |
| 85 | 1-CH₃-2-CH₃-5-CH₃-pyrazol-4-yl | 0 | 2 | H |
| 86 | 6-CF₃-8-Cl-imidazo-[1,2-a]-pyridyl-2-yl | 0 | 2 | H |
| 87 | Benzothiazol-2-yl | 0 | 2 | H |
| 88 | 5-CH₃-benzothiazol-2-yl | 0 | 2 | H |
| 89 | Benzothien-2-yl | 0 | 2 | H |
| 90 | Benzofuran-2-yl | 0 | 2 | H |
| 91 | 1-CH₃-3-CF₂H-pyrazol-4-yl | 0 | 4 | H |
| 92 | 1-CH₃-3-CF₂H-pyrazol-4-yl | 0 | 1 | F |
| 93 | 1-CH₃-3-CF₂H-pyrazol-4-yl | 0 | 1 | H |
| 94 | 2-CF₃-pyridyl-3-yl | 0 | 4 | H |
| 95 | 2-CF₃-pyridyl-3-yl | 0 | 1 | F |
| 96 | 2-CF₃-pyridyl-3-yl | 0 | 1 | H |
| 97 | 2-CH₃-4-CF₂H-thiazol-5-yl | 0 | 4 | H |
| 98 | 2-CH₃-4-CF₃-thiazol-5-yl | 0 | 4 | H |
| 99 | 5-CH₃-1,3,4-thiadiazol-2-yl | 0 | 4 | H |
| 100 | 5-CH₃-1,3,4-oxadiazol-2-yl | 0 | 4 | H |
| 101 | 5-iPr-1,3-oxazol-4-yl | 0 | 2 | H |
| 102 | 2-CH₃-5-CF₃-oxazol-4-yl | 0 | 2 | H |
| 103 | 2-CF₃-thiazol-4-yl | 0 | 2 | H |
| 104 | 2-iPr-thiazol-4-yl | 0 | 2 | H |
| 105 | 2-iPr-4-CH₃-1,3-thiazol-5-yl | 0 | 2 | H |
| 106 | Oxazol-5-yl | 0 | 2 | H |
| 107 | 2-CF₃-pyridyl-3-yl | 0 | 2 | H |
| 108 | 1-CH₃-3-CF₃-pyrazol-4-yl | 0 | 2 | H |
| 109 | 2-Cl-furan-3-yl | 0 | 2 | H |
| 110 | 5-iPr-1,3,4-thiadiazol-2-yl | 0 | 2 | H |
| 111 | 1-CH₃-2-CH₃-5-CH₃-pyrazol-4-yl | 0 | 2 | H |
| 112 | 6-CF₃-8-Cl-imidazo-[1,2-a]-pyridyl-2-yl | 0 | 2 | H |
| 113 | Benzothiazol-2-yl | 0 | 2 | H |
| 114 | 5-CH₃-benzothiazol-2-yl | 0 | 2 | H |
| 115 | Benzothien-2-yl | 0 | 2 | H |
| 116 | Benzofuran-2-yl | 0 | 2 | H |
| 117 | 5-Cl-thiazol-2-yl | 0 | 2 | H |
| 118 | 1-CH₃-3-CF₃-pyrazol-4-yl | 0 | 1 | F |
| 119 | 1-CH₃-3-CF₃-pyrazol-4-yl | 0 | 1 | H |
| 120 | 1-CH₃-3-CF₃-pyrazol-4-yl | 0 | 4 | H |
| 121 | 1-CH₃-3-CF₃-pyrazol-4-yl | 0 | 2 | F |
| 122 | 2-Ph-4-CH₃-1,3-thiazol-5-yl | 0 | 2 | F |
| 123 | 6-CF₃-pyridin-3-yl | 0 | 2 | F |
| 124 | 2-CF₃-6-Cl-pyridin-3-yl | 0 | 2 | F |
| 125 | 4-CF₃-pyridin-3-yl | 0 | 2 | F |
| 126 | Pyrimidin-2-yl | 0 | 2 | F |
| 127 | Pyrimidin-5-yl | 0 | 2 | F |
| 128 | Pyrimidin-4-yl | 0 | 2 | F |
| 129 | 5-Me-1,2-oxazol-3-yl | 0 | 2 | F |
| 130 | 4,5-diCl-l,2-thiazol-3-yl | 0 | 2 | F |
| 131 | Pyridin-3-yl | 2 | 2 | F |
| 132 | Pyridin-3-yl | 1 | 2 | F |
| 133 | 6-CF₃-pyridin-3-yl | 1 | 2 | F |
| 134 | 6-CF₃-pyridin-3-yl | 2 | 2 | F |
| 135 | 4-CF₃-pyridin-3-yl | 2 | 2 | F |
| 136 | Pyrimidin-2-yl | 1 | 2 | F |
| 137 | Pyrimidin-5-yl | 1 | 2 | F |
| 138 | Pyrimidin-4-yl | 1 | 2 | F |
| 139 | Pyrimidin-2-yl | 2 | 2 | F |
| 140 | Pyrimidin-5-yl | 2 | 2 | F |
| 141 | Pyrimidin-4-yl | 2 | 2 | F |

3. The ester according to claim 1, wherein Het, n, m, and X in formula (I) have the meanings indicated in the following Table

| Comp. Nr. | Het | n | m | X |
|---|---|---|---|---|
| 2 | 2-CH₃-4-CF₂H-thiazol-5-yl | 0 | 2 | F |
| 3 | 2-CH₃-4-CF₃-thiazol-5-yl | 0 | 2 | F |
| 4 | 5-CH₃-1,3,4-thiadiazol-2-yl | 0 | 2 | F |
| 5 | 5-CH₃-1,3,4-oxadiazol-2-yl | 0 | 2 | F |
| 6 | 5-iPr-1,3-oxazol-4-yl | 0 | 2 | F |
| 7 | 2-CH₃-5-CF₃-oxazol-4-yl | 0 | 2 | F |
| 8 | 2-CF₃-thiazol-4-yl | 0 | 2 | F |
| 9 | 2-iPr-thiazol-4-yl | 0 | 2 | F |
| 10 | 2-iPr-4-CH₃-1,3-thiazol-5-yl | 0 | 2 | F |
| 11 | Oxazol-5-yl | 0 | 2 | F |
| 12 | 2-CF₃-pyridyl-3-yl | 0 | 2 | F |
| 13 | 1-CH₃-3-CF₂H-pyrazol-4-yl | 0 | 2 | F |
| 14 | 2-Cl-furan-3-yl | 0 | 2 | F |
| 15 | 5-iPr-1,3,4-thiadiazol-2-yl | 0 | 2 | F |
| 16 | 1-CH₃-2-CH₃-5-CH₃-pyrazol-4-yl | 0 | 2 | F |
| 17 | 6-CF₃-8-Cl-imidazo-[1,2-a]-pyridyl-2-yl | 0 | 2 | F |
| 18 | Benzothiazol-2-yl | 0 | 2 | F |
| 19 | 5-CH₃-benzothiazol-2-yl | 0 | 2 | F |
| 20 | Benzothien-2-yl | 0 | 2 | F |
| 21 | Benzofuran-2-yl | 0 | 2 | F |
| 92 | 1-CH₃-3-CF₂H-pyrazol-4-yl | 0 | 1 | F |
| 95 | 2-CF₃-pyridyl-3-yl | 0 | 1 | F |
| 118 | 1-CH₃-3-CF₃-pyrazol-4-yl | 0 | 1 | F |
| 121 | 1-CH₃-3-CF₃-pyrazol-4-yl | 0 | 2 | F |
| 127 | Pyrimidin-5-yl | 0 | 2 | F |
| 129 | 5-Me-1,2-oxazol-3-yl | 0 | 2 | F |
| 131 | Pyridin-3-yl | 2 | 2 | F |

4. Use of an ester of heterocyclic compounds having the general formula (I) according to claim 1, for the control of nematodes.

5. Use of an ester of heterocyclic compounds having the general formula (I) according to claim 4 for the control of Pratylenchus spp, Globodera spp, Heterodera spp, Meloidogyne spp, Aphelenchoides spp, Radopholus Similis, Ditylenchus Dipsaci, Tylenchulus Semipenetrans, Longidorus spp, Xiphinema spp, Trichodorus spp, Bursaphelenchus spp, Belonolaimus spp, of a curative and/or preventive nature.

6. An agronomic composition comprising one or more compounds having Formula (I) according to claim 1, combined with a solvent and/or a solid, liquid or liquefied diluent, optionally one or more surfactants and other agronomically acceptable co-formulants.

7. The agronomic composition according to claim 6, wherein the concentration of active compound having Formula (I) ranges from 0.1 to 90% by weight with respect to the total weight of the composition.

8. The agronomic composition according to claim 6, comprising at least one compound having Formula (I) and at least a second active ingredient selected from insecticides, acaricides, nematocides other than those having Formula (I), herbicides, fungicides, bactericides, fertilizers and biostimulants.

9. The agronomic composition according to claim 8, wherein the weight ratio between the compound having Formula (I) and other active ingredients ranges from 1:100 to 100:1.

10. The agronomic composition according to claim 9, wherein the concentration of active ingredient ranges from 0.5 to 90% by weight with respect to the total weight of the composition.

11. Use of the agronomic composition according to claim 6, for the control of nematodes.

12. Use of the agronomic composition according to claim 11, comprising applying the composition to the crop via the leaves, or to the soil by means of fertigation, or incorporation in the ground, or through seed tanning.

13. A method for the control of nematodes in cultivated areas, comprising applying to any part of the plant to be protected, effective and non-phytotoxic doses of a compound having general formula (I) according to claim 1 and, optionally, one or more further known active ingredients compatible therewith.

14. The agronomic composition according to claim 6, wherein the concentration of active compound having Formula (I) ranges from 0.5 to 90% by weight with respect to the total weight of the composition.

15. The agronomic composition according to claim 8, wherein the weight ratio between the compound having Formula (I) and other active ingredients ranges from 1:10 to 10:1.

16. The agronomic composition according to claim 9, wherein the concentration of active ingredient ranges from 5 to 90% by weight with respect to the total weight of the composition.

17. A method for the control of nematodes in cultivated areas, comprising applying to any part of the plant to be protected, effective and non-phytotoxic doses of a compound according to claim 6, comprising at least one compound having Formula (I) and, optionally, one or more further known active ingredients compatible therewith.
